(19) United States
(12) Patent Application Publication
(10) Pub. No.: US 2022/0105390 A1
(43) Pub. Date: Apr. 7, 2022

(54) REHABILITATION ASSISTANCE SYSTEM, REHABILITATION ASSISTANCE METHOD, AND PROGRAM

(71) Applicant: TOYOTA JIDOSHA KABUSHIKI KAISHA, Toyota-shi Aichi-ken (JP)

(72) Inventors: Hisataka Yuasa, Nagoya-shi Aichi-ken (JP); Issei Nakashima, Toyota-shi Aichi-ken (JP); Takeru Fukagawa, Chiryu-shi Aichi-ken (JP); Takuya Iwata, Toyota-shi Aichi-ken (JP); Takashi Shimizu, Nisshin-shi Aichi-ken (JP); Yohei Otaka, Kariya-shi Aichi-ken (JP); Satoshi Hirano, Nagoya-shi Aichi-ken (JP); Masaki Katoh, Nagoya-shi Aichi-ken (JP); Daisuke Imoto, Nagoya-shi Aichi-ken (JP)

(73) Assignee: TOYOTA JIDOSHA KABUSHIKI KAISHA, Toyota-shi Aichi-ken (JP)

(21) Appl. No.: 17/487,134

(22) Filed: Sep. 28, 2021

(30) Foreign Application Priority Data
Oct. 2, 2020 (JP) 2020-168110

(51) Int. Cl. A63B 24/00; G09B 5/02; G16H 20/30; A63B 71/06

(52) U.S. Cl. CPC A63B 24/0075; G09B 5/02; A63B 22/02; A63B 71/0622; A63B 24/0062; G16H 20/30

(57) ABSTRACT

A rehabilitation assistance system, a rehabilitation assistance method, and a program capable of appropriately assisting the rehabilitation are provided. A rehabilitation assistance system according to an embodiment includes: a first acquisition unit configured to acquire first information in accordance with a level of achievement of rehabilitation training performed by a rehabilitation patient in a first training apparatus; a second acquisition unit configured to acquire second information in accordance with a level of achievement of rehabilitation training performed by the rehabilitation patient in a second training apparatus which is different from the first training apparatus; and a display unit configured to display the first information and the second information in such a way that they are arranged close to each other.

### CROSS REFERENCE TO RELATED APPLICATIONS

This application is based upon and claims the benefit of priority from Japanese patent application No. 2020-168110, filed on Oct. 2, 2020, the disclosure of which is incorporated herein in its entirety by reference.

### BACKGROUND

The present disclosure relates to a rehabilitation assistance system, a rehabilitation assistance method, and a program.

Patent Literature 1 (Japanese Unexamined Patent Application Publication No. 2018-79308) discloses a rehabilitation assistance apparatus that changes a mode of training in accordance with a level of achievement of a rehabilitation exercise and updates the mode of training.

### SUMMARY

There has been a demand, when a rehabilitation patient performs the rehabilitation, to assist the rehabilitation more appropriately.

The present disclosure has been made in view of the aforementioned circumstances, and aims to provide a rehabilitation assistance system, a rehabilitation assistance method, and a program capable of appropriately assisting the rehabilitation.

A rehabilitation assistance system according to one embodiment of the present disclosure includes: a first acquisition unit configured to acquire first information in accordance with a level of achievement of rehabilitation training performed by a rehabilitation patient in a first training apparatus; a second acquisition unit configured to acquire second information in accordance with a level of achievement of rehabilitation training performed by the rehabilitation patient in a second training apparatus which is different from the first training apparatus; and a display unit configured to display the first information and the second information in such a way that they are arranged close to each other.

In the above rehabilitation assistance system, a plurality of items indicating the level of achievement may be set in the first training apparatus, the first acquisition unit may acquire data on the plurality of items as the first information, and the display unit may display data on the plurality of items in a polygonal shape centered on a reference point.

In the above rehabilitation assistance system, the data may be displayed by changing a distance from the reference point in accordance with the level of achievement.

In the above rehabilitation assistance system, the rehabilitation patient may perform rehabilitation training in the first training apparatus a plurality of number of times, and the display unit may display data so as to indicate a time change of the first information.

In the above rehabilitation assistance system, abnormal actions of the rehabilitation patient can be detected in the first training apparatus, and the first information may include the number of times the abnormal actions are detected.

A rehabilitation assistance method according to this embodiment may include: acquiring first information in accordance with a level of achievement of rehabilitation training performed by a rehabilitation patient in a first training apparatus; acquiring second information in accordance with a level of achievement of rehabilitation training performed by the rehabilitation patient in a second training apparatus which is different from the first training apparatus; and displaying the first information and the second information in such a way that they are arranged close to each other.

In the above rehabilitation assistance method, a plurality of items indicating the level of achievement may be set in the first training apparatus, data on the plurality of items may be acquired as the first information, and data on the plurality of items may be displayed in a polygonal shape centered on a reference point.

In the above rehabilitation assistance method, data may be displayed by changing a distance from the reference point in accordance with the level of achievement.

In the above rehabilitation assistance method, the rehabilitation patient may perform rehabilitation training in the first training apparatus a plurality of number of times, and the display unit may display data so as to indicate a time change of the first information.

In the above rehabilitation assistance method, abnormal actions of the rehabilitation patient can be detected in the first training apparatus, and the first information may include the number of times abnormal actions are detected.

A program according to this embodiment causes a computer to execute the above rehabilitation assistance method.

The aim of the present disclosure is to provide a rehabilitation assistance system, a rehabilitation assistance method, and a program capable of appropriately assisting the rehabilitation.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration of a rehabilitation system;

FIG. 2 is a table showing data collected by a rehabilitation assistance apparatus;

FIG. 3 is a diagram showing one example of a display screen of the rehabilitation assistance apparatus; and

FIG. 4 is a flowchart showing a method in the rehabilitation assistance apparatus.

### DESCRIPTION OF EMBODIMENTS

Hereinafter, the present disclosure will be explained with reference to an embodiment of the present disclosure. However, the disclosure set forth in the claims is not limited to the following embodiment. Further, not all the structures explained in the embodiment may be necessary for solving the problem. For a purpose of clarifying the description, the following description and the drawings will be omitted and simplified as appropriate. Throughout the drawings, the same components are denoted by the same reference symbols and overlapping descriptions will be omitted as necessary.

### EMBODIMENTS

Hereinafter, with reference to the drawings, an embodiment of the present disclosure will be described. A rehabilitation training system according to this embodiment performs processing for enabling a trainee to carry out a plurality of kinds of training for the purpose of recovering functions of his/her physical actions. The trainee here is, for example, a person suffering from paralysis in a part of his/her body (this person is also called a rehabilitation patient), which is caused by a stroke (cerebrovascular disease) or a spinal cord injury. The trainee's physical action functions have been impaired due to the above disease. In this embodiment, physical actions are, for example, actions included in Activities of Daily Living (ADL) and include actions such as eating, toileting, dressing, walking, etc.

A trainee carries out one or a plurality of kinds of training in order to recover the functions of his/her impaired physical actions. To be specific, for example, training for recovering functions of a physical action eating includes training of a meal intake action, training of a swallowing action, and the like. Further, the training for recovering functions of the toileting action includes, for example, training of a moving action for moving to each of a bed, a wheelchair, and a toilet seat, training for toileting, and the like. Further, the training for recovering the function of the walking action includes, for example, training for maintaining the sitting posture, training for maintaining the standing posture, training of flat ground walking, training of walking on a slope, and the like. Further, the training includes, besides the training in which the trainee actively carries out physical actions as described above, passive training such as a massage in which the trainee receives stimulation to his/her joints or muscles from a helper. The aforementioned training is also referred to as rehabilitation or a rehabilitation training.

Some training performed using the rehabilitation training system may require assistance by therapists. Therapists are persons who have expertise that is necessary to assist trainees. In this embodiment, the therapists include a physical therapist, an occupational therapist, and a speech therapist. The therapists assist training performed by the trainees in accordance with expertise that they each have.

The physical therapist is a person who offers a physical therapy for persons who have physical disabilities mainly for the purpose of recovering fundamental action abilities. The physical therapy is a therapeutic treatment performed on persons whose functions of physical actions have been impaired due to diseases, injuries, disabilities and the like for the purpose of maintaining or improving these functions. The physical therapist carries out a physical therapy by using exercises, heat, electricity and the like. The physical therapist is also referred to as a PT.

The occupational therapist is a person who offers an occupational therapy for persons who have physical or mental disabilities mainly for the purpose of recovering their applicative action abilities or social adaptability. The “occupation” here includes daily activities such as bathing, dressing, and toileting. The occupational therapist is also referred to as an OT.

The speech therapist is a person who carries out language training and other training exercises and provides tests, advice, guidance, and other assistance necessary for the above training exercises for persons who have disabilities in their speech functions, language functions, or hearing for the purpose of maintaining and improving these functions. The speech therapist further conducts training such as meal intake or swallowing under the instructions of doctors or dentists. The speech therapist is also referred to as a speech-language-hearing therapist or an ST. Hereinafter, persons such as the PT, the OT, and the ST who assist rehabilitation training performed by trainees are also called assistants. The assistants may include doctors, nurses and so on.

The rehabilitation training system helps an assistant set a goal for a trainee. For example, the rehabilitation training system is a rehabilitation assistance apparatus (this apparatus is also referred to as a rehabilitation assistance system) that collects data regarding rehabilitation training conducted by a plurality of trainees. Then the assistance apparatus refers to data of past rehabilitation patients and displays a display screen which enables rehabilitation to be assisted. A display unit of the rehabilitation assistance apparatus displays a screen for enabling the goal for the trainee (target patient) to be set. For example, the assistant and the trainee are able to set a goal to be achieved at a predetermining timing such as at the time of discharge while looking at the screen. The assistant or the trainee is able to make a rehabilitation plan in accordance with the goal.

### First Embodiment

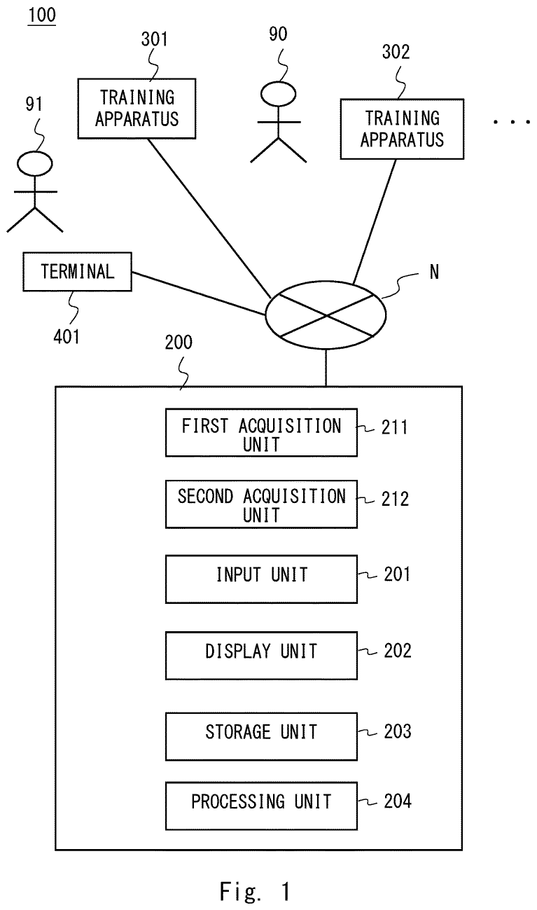

With reference to FIG. 1, an example of a rehabilitation training system (this system is simply referred to as a training system) will be described. A training system 100 includes an assistance apparatus 200, training apparatuses 301 and 302, and a terminal 401. In the following description, the training apparatus 301 may be referred to as a first training apparatus and the training apparatus 302 may be referred to as a second training apparatus. While the training system 100 includes two training apparatuses 301 and 302 in FIG. 1, the training system 100 may include three or more training apparatuses. Further, a plurality of rehabilitation patients 90 may perform training using the training apparatuses 301 and 302. That is, the rehabilitation training system 100 may include a third training apparatus and a fourth training apparatus whose types are different from those of the training apparatuses 301 and 302.

It is assumed here that the training apparatuses 301 and 302 are training equipment for providing walking training. That is, the rehabilitation patient 90 performs rehabilitation training for improving his/her walking functions using the training apparatuses 301 and 302. The training apparatuses 301 and 302 are, for example, Welwalk WW-1000 or WW-2000. Further, the training apparatus 301 is training equipment for enabling a user to perform walking training on a treadmill. Further, the rehabilitation training system 100 may include a training apparatus for enabling a user to perform indoor walking or applied walking.

As a matter of course, the rehabilitation training is not limited to walking training and may instead be training of contacting or toileting. The training apparatuses 301 and 302 include drive equipment such as a motor, or a sensor. The training apparatuses 301 and 302 are installed in a rehabilitation facility of a hospital or the like. As a matter of course, the training apparatuses 301 and 302 may be equipment installed in the house of the rehabilitation patient 90. While only one rehabilitation patient 90 is shown in FIG. 1, a plurality of rehabilitation patients 90 may perform rehabilitation training.

The terminal 401 is a device that enables an assistant 91 or the like to input data. The terminal 401 is, for example, a tablet computer, a smartphone, a personal computer or the like. The assistant 91 is able to input data regarding the rehabilitation patient 90 or rehabilitation training performed by the rehabilitation patient 90 using the terminal 401. The terminal 401 may be a device attached to the training apparatus 301 or the training apparatus 302. The rehabilitation training system 100 may include a plurality of terminals 401. As described above, the assistant 91 is a PT, an OT, an ST, a doctor, or a nurse.

The assistance apparatus 200 is a device to/from which data can be input/output. The assistance apparatus 200 includes an input unit 201, a display unit 202, a storage unit 203, a processing unit 204, a first acquisition unit 211, and a second acquisition unit 212. The assistance apparatus 200 may be, like the terminal 401, a tablet computer, a smartphone, a personal computer or the like. The assistance apparatus 200 may use some of the functions of the terminal 401. The assistance apparatus 200 serves as a rehabilitation assistance system that assists rehabilitation training.

The first acquisition unit 211 acquires data from the training apparatus 301. The second acquisition unit 212 acquires data from the training apparatus 302. The first acquisition unit 211 acquires, for example, first information in accordance with a level of achievement of rehabilitation training performed by a rehabilitation patient in the training apparatus 301. The second acquisition unit 212 acquires second information in accordance with the level of achievement of the rehabilitation training performed by the rehabilitation patient in the training apparatus 302.

Further, the first acquisition unit 211 and the second acquisition unit 212 may acquire data from the terminal 401. The training apparatuses 301 and 302, the terminal 401, and the assistance apparatus 200 are connected to one another in such a way that they can communicate with one another via a network N. The network N is a communication line network such as the Internet, an intranet, a mobile telephone network, or a Local Area Network (LAN).

The acquisition of data by the first acquisition unit 211 and the second acquisition unit 212 is not limited to the one that uses the network N. For example, at least a part of data may be acquired via a USB memory or the like. The data may be acquired by wireless communication such as WiFi (registered trademark) or by wired communication. The first acquisition unit 211 and the second acquisition unit 212 include a wireless or wired data input/output interface. The data acquisition may be automatically performed by the first acquisition unit 211 and the second acquisition unit 212. The assistant 91 may select necessary data and write the data into the assistance apparatus 200.

The storage unit 203 stores various kinds of data acquired by the first acquisition unit 211 and the second acquisition unit 212. The storage unit 203 includes a non-volatile memory such as a hard disc drive, a flash memory, an Erasable Programmable Read Only Memory (EPROM) or a Solid State Drive (SSD).

The input unit 201 accepts an input from the assistant 91 or the rehabilitation patient 90. The input unit 201 includes an input device such as a touch panel, a keyboard, a mouse or the like. Furthermore, a voice input using a microphone may be performed.

The display unit 202 displays a predetermined screen for the assistant 91 or the rehabilitation patient 90. The display unit 202 includes, for example, a display device such as a liquid crystal display. The display unit 202 may further include a display control circuit or the like of the display device.

The processing unit 204 performs processing by referring to the data stored in the storage unit 203.

The assistance apparatus 200 is not limited to a physically single apparatus. For example, the display unit 202 and the input unit 201 may serve as a main body apparatus of a tablet PC or the like and the storage unit 203 may serve as a storage device that is provided separately from the main body apparatus. Then the tablet PC may acquire the data from the storage unit 203 using a wireless LAN such as WiFi (registered trademark).

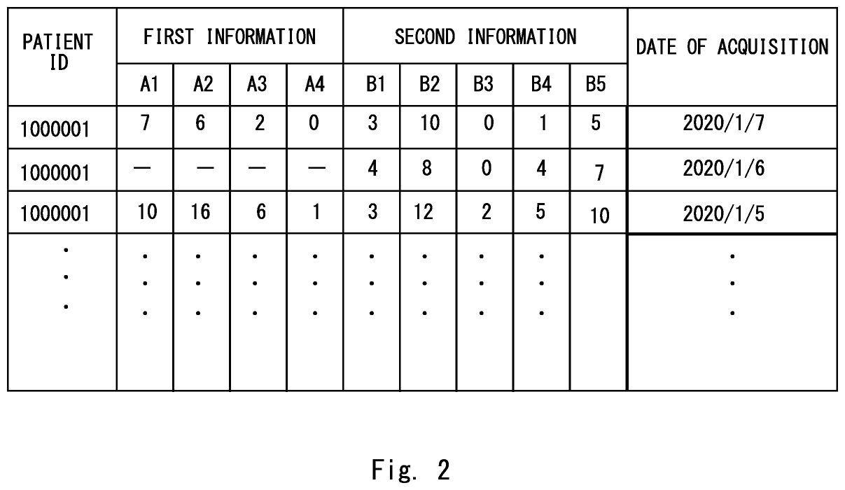

The storage unit 203 includes a database that stores first and second information regarding rehabilitation patients. That is, the storage unit 203 stores, for each rehabilitation patient, the first and second information. With reference to FIG. 2, one example of the data stored in the storage unit 203 will be described. FIG. 2 is a table showing a list of the first and second information regarding a rehabilitation patient. FIG. 2 is a list of data indicating information on one patient.

As shown in FIG. 2, the storage unit 203 stores information for each patient as a database. Four items A1-A4 are set as the first information. Five items B1-B5 are set as the second information. The items A1-A4 indicate the level of achievement of the training in the training apparatus 301. The items B1-B5 indicate the level of achievement of the training in the training apparatus 302 that is different from the training apparatus 301. The items A1-A4 may be items that are different from the items B1-B5 or at least some of them may be the same.

For example, the items A1-A4 are data acquired at the time of training in the training apparatus 301. Specifically, the data in the items A1-A4 indicate results of detection in various types of sensors provided in the training apparatus 301 or values obtained from the results of the detection. For example, the training apparatus 301 determines whether or not actions of the rehabilitation patient 90 match preset abnormal actions based on the results of the detection in the various types of sensors. Then the training apparatus 301 obtains the number of times each of the abnormal actions has been detected.

The data pieces in the items A1-A4 each indicate the number of times abnormal actions are detected. Therefore, as the functions are improved by the rehabilitation training, the number of times abnormal actions are detected in the training apparatus 301 decreases. In other words, the data in the items A1-A4 indicates the level of achievement of the training in the training apparatus 301.

The items B1-B5 are data acquired at the time of training in the training apparatus 302. Specifically, the data in the items B1-B5 indicates results of detection in various types of sensors provided in the training apparatus 302 or values obtained from the results of the detection. For example, the training apparatus 302 determines whether or not actions of the rehabilitation patient 90 match preset abnormal actions based on the results of the detection in the various types of sensors. Then the training apparatus 302 obtains the number of times each of the abnormal actions has been detected.

The data pieces in the items B1-B5 each indicate the number of times abnormal actions are detected. Therefore, as the functions are improved by the rehabilitation training, the number of times abnormal actions are detected in the training apparatus 302 decreases. In other words, the data in the items B1-B5 indicates the level of achievement of the training in the training apparatus 302. The second acquisition unit 212 acquires data on the plurality of items as the second information.

The items A1-A4 and the items B1-B5 are associated with the date of acquisition of the data. That is, the date of training when the rehabilitation patient 90 has performed training is the date of acquisition of the data. Then the data in each item is stored for each date of training. Regarding the dates when the rehabilitation patient 90 does not perform training using the training apparatus 301, data in the items A1-A4 is left blank.

The first acquisition unit 211 acquires data on a plurality of items as the first information. The first acquisition unit 211 acquires the first information for each training. The second acquisition unit 212 acquires data on a plurality of items as the second information. The second acquisition unit 212 acquires second information for each training. Then the storage unit 203 stores the first and second information in association with the date of acquisition such as the date of training. Further, when the rehabilitation patient 90 performs training twice or more times in one day using one training apparatus 301, besides the date of acquisition (the date of training), information such as the time of the acquisition (training time) may be associated with the above information.

As described above, one rehabilitation patient 90 performs training using training apparatuses 301 and 302 of two or more types. In this case, items set in the training apparatus 301 and items set in the training apparatus 302 are different from each other. The first acquisition unit 211 acquires data indicating the level of achievement of training from the training apparatus 301 as the first information. The second acquisition unit 212 acquires data indicating the level of achievement of training from the training apparatus 302 as the second information. The storage unit 203 may store the first and second information for each rehabilitation patient.

The first and second information are not limited to the number of times abnormal actions are detected in the training apparatuses 301 and 302, respectively. The first and second information may be indicated, for example, by a time or a percentage (%). Alternatively, the first and second information may be indicated, for example, by a load or an angle detected by a load sensor or an angle sensor. Otherwise the first and second information may be indicated by a joint angle of a body detected in a posture angle sensor. That is, it is sufficient that the first and second information be data obtained directly or indirectly by a sensor.

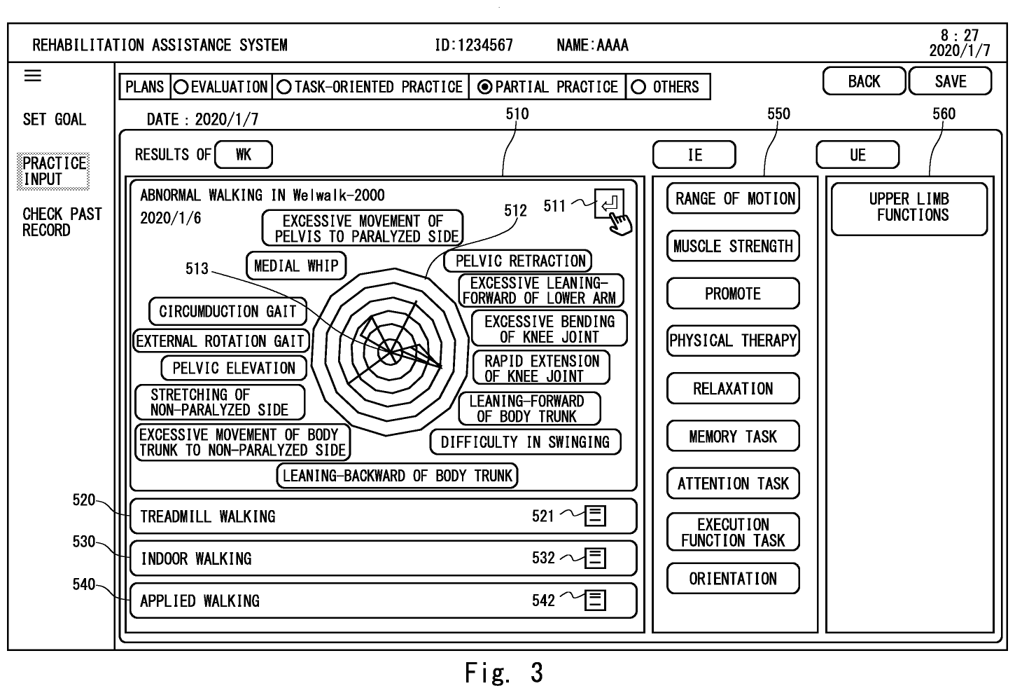

FIG. 3 is a diagram showing one example of a display screen in the display unit 202. FIG. 3 is a diagram showing a display example when training is performed in training apparatuses of four types. Specifically, the display screen displays a first display region 510, a second display region 520, a third display region 530, a fourth display region 540, a fifth display region 550, and a sixth display region 560.

The first display region 510 displays the first information from WelWalk-2000, which is the first training apparatus. The second display region 520 displays the second information from the second training apparatus that provides walking training using a treadmill. The third display region 530 displays third information from the third training apparatus that provides indoor walking training. The fourth display region 540 displays fourth information from the fourth training apparatus that provides applied walking training.

The first display region 510, the second display region 520, the third display region 530, and the fourth display region 540 respectively include detail display buttons 511, 521, 532, and 542. By clicking the detail display buttons 511, 521, 532, and 542, details of the information are displayed. FIG. 3 shows the first information in the first display region 510 in detail. When the assistant 91 clicks the detail display button 511, the first display region 510 becomes wider. When the assistant 91 clicks the detail display button 511 once again, the first display region 510 is folded and becomes narrower.

The display unit 202 may display the first information and the second information in such a way that they are arranged close to each other (side by side). The first information and the second information may be displayed in a graph or a radar chart independently from each other. That is, the first information and the second information may be displayed in different regions (display windows) on a display screen. Alternatively, the first information and the second information may be displayed in one graph or one radar chart. The first information and the second information may be displayed in a common region (display window) of the display screen in a superimposed manner. Alternatively, the mode in which the first and second information are displayed in such a way that they are arranged close to each other is not limited to the above display mode. For example, the display unit 202 may concurrently display the first and second information. The display unit 202 may concurrently display the first and second information in, for example, one graph. The display unit 202 may concurrently display the first and second information in graphs different from each other. Alternatively, the display unit 202 may display the first and second information in graphs of different forms.

The first display region 510 will be described in detail. The level of achievement of training in the training apparatus 301 is evaluated in a plurality of items. Then the data regarding the plurality of items is indicated by a radar chart 512 having a circular shape or a polygonal shape.

Specifically, the training apparatus 301 detects 14 abnormal walking actions (e.g., leaning-forward of body trunk, difficulty in swinging, leaning-backward of body trunk, etc.) during training. The first acquisition unit 211 acquires the number of times abnormal walking actions are detected in one rehabilitation training. The display unit 202 plots the number of times of detection on a tetradecagon centered on a reference point 513. When the number of times abnormal walking actions are detected is zero, a point indicating the number of times of detection is plotted on the reference point 513. Then points indicating the number of times of detection are plotted in such a way that the distance from the reference point 513 increases as the number of times of detection increases. Therefore, the display unit 202 displays the information in such a way that the distance from the reference point 513 is changed in accordance with the level of achievement.

Further, when the rehabilitation patient 90 performs training in the training apparatus 301 a plurality of times, the display unit 202 may display the information so as to indicate a time change of the first information. For example, data acquired in the latest five training sessions may be displayed. Then the display unit 202 displays the information while changing the shapes or the colors of the points for each date of training (each date of acquisition). In this way, by displaying levels of achievement of a plurality of training sessions at one time, it is possible to check the degree of progress of the training. Further, the levels of achievement may be displayed in chronological order. The assistant 91 may specify the date and the time when the data to be displayed has been acquired and the range of the data to be displayed.

The information indicating the level of achievement in each training apparatus is displayed in each display region. The number of display regions displayed on the display screen may be changed in accordance with the number of training apparatuses where training has been performed. For example, regarding a rehabilitation patient who performs training only in the two training apparatuses 301 and 302, the display unit 202 may display only two display regions of the first display region 510 and the second display region 520. That is, since the information from the third and fourth training apparatuses is not acquired, the display in the third display region 530 and the fourth display region 540 can be omitted. It is therefore possible to change the number of display regions for each patient.

In the second display region 520, the third display region 530, and the fourth display region 540, information in accordance with the level of achievement in each training apparatus is displayed. The second display region 520 displays the second information in accordance with the level of achievement in the second training apparatus. The third display region 530 displays the third information in accordance with the level of achievement in the third training apparatus, and the fourth display region 540 displays the fourth information in accordance with the level of achievement in the fourth training apparatus.

For example, each of the training apparatuses detects abnormal actions such as abnormal walking actions of the rehabilitation patient 90. Each of the training apparatuses transmits the number of times abnormal actions are detected to the assistance apparatus 200. The display unit 202 displays the number of times of detection as a radar chart. As a matter of course, the display unit 202 may display information other than the abnormal actions. The display unit 202 may concurrently display the details of the information obtained in two or more training apparatuses. Further, the display unit 202 may display information obtained in two or more training apparatuses on one radar chart 512.

Training menus are displayed in the fifth display region 550 and the sixth display region 560. For example, training menus for improving reduced abilities (IE) are displayed in the fifth display region 550 and training menus for improving upper limb functions (UE) are displayed in the sixth display region 560. Further, the assistance apparatus 200 may present efficient training menus in accordance with the level of achievement of training performed by the rehabilitation patient. For example, the processing unit 204 determines efficient training menus based on at least one of the first information and the second information. The display unit 202 highlights the efficient training menus.

The training menus displayed in the fifth display region 550 and the sixth display region 560 may be changed for each training apparatus or may be common among a plurality of training apparatuses.

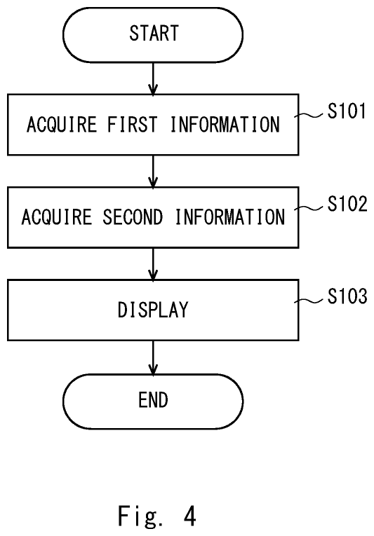

With reference to FIG. 4, a method in the assistance apparatus 200 will be described. First, the first acquisition unit 211 acquires the first information (S101). In this example, for example, data acquired in the first training apparatus 301 is acquired as the first information. Next, the second acquisition unit 212 acquires the second information from the second training apparatus 302 (S102). In this example, for example, the data acquired in the second training apparatus 302 is acquired as the second information. Then the display unit 202 displays the first information and the second information (S103). As described above, the display unit 202 displays the first information and the second information in such a way that they are arranged close to each other (next to each other).

Further, a part or all of the processing in the aforementioned systems, apparatuses, terminals and the like may be provided as a computer program. The program can be stored and provided to a computer using any type of non-transitory computer readable media. Non-transitory computer readable media include any type of tangible storage media. Examples of non-transitory computer readable media include magnetic storage media (such as flexible disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Note that the present disclosure is not limited to the above embodiments and may be changed as appropriate without departing from the spirit of the present disclosure.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A rehabilitation assistance system comprising:
a first acquisition unit configured to acquire first information in accordance with a level of achievement of rehabilitation training performed by a rehabilitation patient in a first training apparatus;
a second acquisition unit configured to acquire second information in accordance with a level of achievement of rehabilitation training performed by the rehabilitation patient in a second training apparatus which is different from the first training apparatus; and
a display unit configured to display the first information and the second information in such a way that they are arranged close to each other.

2. The rehabilitation assistance system according to claim 1, wherein
a plurality of items indicating the level of achievement are set in the first training apparatus,
the first acquisition unit acquires data on the plurality of items as the first information, and
the display unit displays data on the plurality of items in a polygonal shape centered on a reference point.

3. The rehabilitation assistance system according to claim 2, wherein the data is displayed by changing a distance from the reference point in accordance with the level of achievement.

4. The rehabilitation assistance system according to claim 1, wherein
the rehabilitation patient performs rehabilitation training in the first training apparatus a plurality of number of times, and
the display unit displays data so as to indicate a time change of the first information.

5. The rehabilitation assistance system according to claim 1, wherein
abnormal actions of the rehabilitation patient can be detected in the first training apparatus, and
the first information includes the number of times the abnormal actions are detected.

6. A rehabilitation assistance method comprising:
acquiring first information in accordance with a level of achievement of rehabilitation training performed by a rehabilitation patient in a first training apparatus;
acquiring second information in accordance with a level of achievement of rehabilitation training performed by the rehabilitation patient in a second training apparatus which is different from the first training apparatus; and
displaying the first information and the second information in such a way that they are arranged close to each other.

7. The rehabilitation assistance method according to claim 6, wherein
a plurality of items indicating the level of achievement are set in the first training apparatus,
data on the plurality of items is acquired as the first information, and
data on the plurality of items is displayed in a polygonal shape centered on a reference point.

8. The rehabilitation assistance method according to claim 7, comprising displaying data by changing a distance from the reference point in accordance with the level of achievement.

9. The rehabilitation assistance method according to claim 6, wherein
the rehabilitation patient performs rehabilitation training in the first training apparatus a plurality of number of times, and
data is displayed so as to indicate a time change of the first information.

10. The rehabilitation assistance method according to claim 6, wherein
abnormal actions of the rehabilitation patient can be detected in the first training apparatus, and
the first information includes the number of times the abnormal actions are detected.

11. A non-transitory computer readable medium storing a program for causing a computer to execute the rehabilitation assistance method according to claim 6.
